(19) United States
(12) Patent Application Publication
(10) Pub. No.: US 2025/0149158 A1
(43) Pub. Date: May 8, 2025

(54) METHOD OF GENERATING A PROTOCOL FOR A MEDICAL IMAGING DEVICE

(71) Applicant: Siemens Healthineers AG, Forchheim (DE)

(72) Inventor: Srikrishna PRASAD, Erlangen (DE)

(73) Assignee: Siemens Healthineers AG, Forchheim (DE)

(21) Appl. No.: 18/937,405

(22) Filed: Nov. 5, 2024

(30) Foreign Application Priority Data
Nov. 7, 2023 (DE) 10 2023 211 015.6

(51) Int. Cl. G16H 40/63; G16H 10/60; H04L 9/00

(52) U.S. Cl. CPC G16H 40/63; G16H 10/60; H04L 9/008

(57) ABSTRACT

A computer-implemented method of generating a protocol for a medical imaging device installed at a specific site, comprises: providing a homomorphic encrypted large-language model trained to generate a homomorphic encrypted medical imaging protocol; obtaining patient-specific input data for an intended medical imaging task; performing homomorphic encryption on the patient-specific input data; applying the homomorphic encrypted patient-specific input data to the homomorphic encrypted large-language model to generate the medical imaging protocol; and performing homomorphic decryption on the homomorphic encrypted medical imaging protocol to obtain a plain-text medical imaging protocol. The homomorphic encryption and decryption utilize a first encryption scheme specific to the site and a second encryption scheme specific to the vendor.

### CROSS-REFERENCE TO RELATED APPLICATION(S)

The present application claims priority under 35 U.S.C. § 119 to German Patent Application No. 10 2023 211 015.6, filed Nov. 7, 2023, the entire contents of which is incorporated herein by reference.

### BACKGROUND

In order to perform a medical imaging procedure so that a satisfactory image is obtained, the imaging device must be configured correctly. The set of input parameters used during an imaging task is referred to as the “imaging protocol” or “medical imaging protocol”. A medical imaging protocol is a predefined procedure or set of guidelines that outlines how to conduct a specific medical imaging examination. These protocols aim to standardize the imaging process to ensure consistency and reproducibility, while also ensuring that the imaging procedure provides the necessary diagnostic information and minimizes patient exposure to potential risks such as radiation. Examples of values that might be specified in a medical imaging protocol include the modality, which refers to the type of imaging technology used, such as X-ray, computed tomography (CT), magnetic resonance imaging (MRI), ultrasound, or positron emission tomography (PET). The protocol also includes specific parameters used by the imaging device. For instance, in an MRI protocol, these might include the magnetic field strength, echo time, repetition time, inversion time, and flip angle. For a CT protocol, parameters could include tube current, tube voltage, rotation time, and slice thickness. Some protocols may specify the use of a contrast agent to enhance the visibility of certain tissues or structures, including the type of contrast agent, the dosage, and the timing of its administration. The protocol may also provide instructions for patient positioning in the scanner and for modalities like MRI, the protocol may specify the use of certain sequences that provide different types of contrast between tissues.

The user of a medical imaging device, for example a radiographer, generally requires extensive training in order to become familiar with the (usually many) features of the device and the various input parameters. To choose an optimal set of imaging protocol parameters for an intended imaging task, the user may refer to relevant or similar prior scans, imaging protocols and patient outcomes. In order to correctly interpret such information, the user may have acquired years of experience.

Even for an experienced user of the imaging device, it can be challenging to define a correct protocol for each imaging task. An incorrect or sub-optimal imaging protocol can result in the need to repeat the imaging task (assuming the protocol deficiencies are detected) and may even have a detrimental effect on patient care.

To improve the step of compiling a protocol, the vendor of the imaging device may provide templates, for example templates for specific imaging tasks (e.g. neck CT; shoulder MRT; etc.). The radiographer can select a suitable protocol and make the necessary adjustments to tailor the protocol to the specific patient and to cross-verify to ensure that the protocol complies with any relevant protocol cookbook, for example a protocol cookbook of that specific facility or clinic.

Furthermore, the lack of standardization between different facilities and the different personal preferences among experienced operators can lead to significant variations in image quality, radiation dose, and diagnostic outcomes, even for the same patient and the same imaging task. Inexperienced operators may find it difficult to put together an optimal protocol or to select the most suitable protocol from a cookbook, and the quality of the resulting diagnostic images can be sub-optimal, with an adverse effect on patient care. The known approaches are therefore time-consuming and error-prone.

One possible way of overcoming these problems might be to establish a collection of satisfactory protocols for a facility, for example in the radiology department of a hospital. Over time, the collection could comprise many thousands of protocols. A radiographer could search this comprehensive “protocol library” in the hope of finding a protocol for a specific imaging task that was created in the past for a similar patient. However, compilation originally submitted such a collection would require a very long time, during which many thousands of imaging tasks would need to be done on many different types of patient in order to establish a sufficiently large database.

In another scenario, several facilities would share their collections of medical imaging protocols. However, this option is effectively ruled out because of the need to ensure patient privacy. Generally, any medical facility ensures that its patient data is stored securely, for example images and protocols may be stored in a local PACS (picture archiving and communication system) that is accessible only to authorized facility staff or to programs installed in that facility.

### SUMMARY

It is an object of one or more embodiments of the present invention to provide a secure and straightforward way of obtaining a medical image protocol.

At least this object is achieved by the claimed computer-implemented methods of generating a protocol for a medical imaging device and of training a large-language model for that purpose, and by the claimed data processing arrangement.

The inventive computer-implemented method of generating a medical imaging protocol is used, for example by a radiographer, to generate a protocol for input to a medical imaging device that is installed at a specific customer site, for example in a hospital or clinic. The inventive computer-implemented method comprises the steps of providing a homomorphic encrypted large-language model trained to generate a homomorphic encrypted medical imaging protocol; obtaining patient-specific input data for an intended medical imaging task; performing homomorphic encryption on the patient-specific input data; applying the homomorphic encrypted patient-specific input data to the trained homomorphic encrypted large-language model. Homomorphic decryption is then performed on the generated homomorphic encrypted medical imaging protocol to obtain a plain-text medical imaging protocol. In the inventive method, homomorphic encryption and decryption are done using a first encryption scheme that is specific to the customer site, and a second encryption scheme that is specific to the vendor of the large-language model. The site-specific and vendor-specific encryption/decryption keys can be stored in an established manner in a key store, for example in an authenticated gateway device.

The inventive computer-implemented method of training a large-language model for use in the above method comprises the steps of modifying a large-language model to comprise a homomorphic encrypted layer architecture; obtaining homomorphic encrypted training data based on imaging protocols for the medical imaging device installed at the specific customer site; and training the homomorphic encrypted large-language model on the homomorphic encrypted training data to generate a homomorphic encrypted medical imaging protocol. Again, homomorphic encryption and decryption are done using the first (i.e. site-specific) encryption scheme and the second (i.e. vendor-specific) encryption scheme.

An advantage of the inventive approach is that it provides a way of using a vendor-provided machine-learning tool to learn from a favorably large collection of medical imaging protocol data, while complying with patient data privacy constraints. The homomorphic encrypted large-language model is provided by the vendor, who may also provide instances of the same large-language model (LLM) to various other customers. The vendor may also provide data processing services to multiple customers. The two-scheme encryption deployed by the inventive method allows data processing steps to be performed at the customer site, but also allows data processing steps to be performed off-site (for example at the vendor site), without compromising patient data privacy. In this way, the inventive method makes it impossible for the vendor (or any other customer of that vendor) to access any patient data at any stage in the inventive method. Sensitive patient data remains on-site (e.g. within a clinic network), and any data that leaves the site is no longer human-readable, i.e. no longer in a form that makes sense to a human reader.

Furthermore, the inventive computer-implemented method can quickly generate an imaging protocol for the user, and can therefore help in optimizing the imaging workflow at the customer site (e.g. a hospital, a clinic or similar facility). The user—for example a radiographer—need only provide basic information such as patient-specific data, a descriptor of the intended imaging task, etc.

The inventive data processing arrangement comprises a means, mechanism, device and/or system for providing a homomorphic encrypted large-language model; a model development stage configured to develop and train the homomorphic encrypted large-language model to create homomorphic encrypted output text from input text; a means, mechanism, device and/or system for obtaining patient-specific input text for an intended medical imaging task; and a protocol generation stage configured to apply the patient-specific input data to the trained homomorphic encrypted large-language model to obtain a plain-text medical imaging protocol for the intended imaging task.

The data processing arrangement can be realised in the form of computer program modules which can run on any suitable platform(s) and which comprise program units to perform the steps of the inventive computer-implemented method. In the context of embodiments of the present invention, a computer program module that has a means, mechanism, device and/or system for receiving the homomorphic encrypted vendor model and the site-specific input data input and a means, mechanism, device and/or system for outputting a trained large-language model is referred to herein as the “model training stage” of the data processing arrangement; a computer program module that has a means, mechanism, device and/or system for receiving the trained homomorphic encrypted large-language model and the patient-specific input data and a means, mechanism, device and/or system for outputting the plain-text medical imaging protocol is referred to herein as a “protocol generation stage” of the data processing arrangement. The inventive data processing arrangement can be a distributed system, with units and modules distributed as appropriate over customer and vendor sites for example.

The inventive computer-implemented method can be used by a clinician such as a radiographer or radiologist. The computer program product can be configured to present the generated imaging protocol to the user in any suitable manner (e.g. to show it on the screen of a device such as a desktop computer, a tablet computer, etc.).

In particular, the plain-text medical imaging protocol generated can be used to control the medical imaging device installed at the specific site. In one embodiment, the medical imaging protocol can be utilized to manage the operations of the medical imaging device, such as initiating, pausing, or terminating imaging procedures. In another embodiment, the medical imaging protocol can be used to adjust the imaging parameters of the device, including but not limited to, the intensity, frequency, and duration of the imaging signals (x-ray signals, magnetic and radiofrequency signals).

At least the object of one or more embodiments of the present invention is also achieved by a non-transitory computer program product with a computer program that is directly loadable into the memory of a data-processing arrangement, and which comprises program units to perform the steps of the inventive computer-implemented method when the program is executed. The computer program product may be stored on a computer-readable data carrier.

Particularly advantageous embodiments and features of the present invention are given by the dependent claims, as revealed in the following description. Features of different claim categories may be combined as appropriate to give further embodiments not described herein.

As used in the context of embodiments of the present invention, the term “patient-specific input data” can refer to any data that is unique to an individual patient and which is used in the context of that patient's healthcare management. Such data is typically used to inform clinical decisions, guide treatment plans, and monitor patient progress, and can be derived from a variety of sources including medical history, physical examinations, laboratory tests, imaging studies, and patient-reported outcomes. Examples of patient-specific input data include demographic Information (including the patient's age, sex, ethnicity, and other socio-economic factors that may influence health outcomes), medical history (which encompasses past and present illnesses, surgeries, allergies, and medication use), physical examination findings (e.g., observations made by healthcare providers during a physical examination, such as heart rate, blood pressure, and findings from the examination of various body systems) and/or laboratory test results (quantitative and qualitative data obtained from the analysis of biological samples, such as blood or urine, including values such as blood glucose levels, cholesterol levels, and complete blood count results). Any patient-specific input data that is used in the compilation of an imaging protocol is generally in the form of words and numbers, and may also be referred to as “patient-specific input text”.

In the context of embodiments of the present invention, a vendor shall be understood to supply the customer with medical imaging devices and also with software for operating the devices. The same vendor may also provide the customer with a data processing arrangement for carrying out the inventive computer-implemented method.

The imaging modality or medical imaging device for which a generated or synthesized protocol is intended may be a CT device, an MRI device, a PET device, a single-photon emission computed tomography (SPECT) device, an X-ray device, etc. The terms “medical imaging protocol”, “imaging protocol” and simply “protocol” may be used interchangeably.

In the context of embodiments of the present invention, expressions such as “machine learning algorithm”, “large language model”, “artificial intelligence tool” and “AI model” shall be understood to be synonyms and may be used interchangeably. A large-language model is an artificial neural network particularly suited for applications which involve understanding of or generation of text. The machine learning algorithm is trained to generate a medical imaging protocol for an intended imaging task, using only a number of patient-specific parameters. Any suitable LLM architecture may be used, for example a long short-term memory (LSTM) network architecture. The chosen LLM architecture is further modified and updated by the vendor to comprise a homomorphic encrypted layer architecture for the highly-secure privacy-preserving machine learning method according to embodiments of the present invention. Further algorithms such as a cross-entropy loss function module and an Adam optimizer module may be implemented. Any algorithms required for the inventive method may be provided by the vendor and/or by a third party.

In a preparatory stage, a homomorphic encrypted version of the vendor model is generated with the customer encryption scheme and the vendor encryption scheme as indicated above. As will be known to the skilled person, homomorphic encryption is a form of encryption that allows computations to be carried out on ciphertext, generating an encrypted result which, when decrypted, matches the result of operations performed on the plaintext. This property allows third-party systems to process data without needing to decrypt it, thereby preserving data privacy and security. The homomorphic encrypted large-language model is referred to as the “HE-LLM” in the following.

In the inventive method, training the HE-LLM comprises the steps of: compiling a training dataset; assigning a ground truth to the training dataset; and applying the HE-LLM to the training data set in order to approximate the ground truth. These steps are repeated for all available training datasets and for a suitable number of epochs until a desired level of accuracy has been achieved.

In the inventive method, training data is based on medical imaging protocols that have been previously created at that customer site (referred to herein as the facility). For example, the facility may maintain a database of all medical imaging protocols for its imaging modalities. If necessary, the protocols are converted to a common format such as a suitable markup language (e.g. XML). Since the accuracy of a machine learning algorithm depends to a large extent on the number of datasets with which it is trained, in a preferred embodiment of the present invention the LLM is trained using at least several hundred medical imaging protocols, more preferably several thousand medical imaging protocols.

The facility may also deploy medical imaging protocol cookbooks to ensure that facility-specific standards are adhered to in each imaging procedure. In a particularly preferred embodiment of the present invention, therefore, the protocol data comprises any relevant medical imaging protocol cookbooks for a medical imaging device. A protocol cookbook in PDF format can be scanned and digitized, and converted to the same format used for the protocols.

The LLM could also be trained with synthetically generated medical imaging protocols and/or protocol cookbooks in order to improve its accuracy. By training the LLM on many thousands of verified or approved protocols used for past imaging tasks, a synthesized protocol can be expected to be very precise, so that the inventive approach can help minimize the radiation dose to which a patient is exposed.

To prepare a training dataset, tokenization and padding is performed on each input dataset. During tokenization, the text of each document is converted into a sequence of tokens, and each token is assigned a number. To ensure that the tokenized sequences have the same length, padding is done as necessary. In a “cleaning” stage, duplicate or irrelevant datasets are removed. Upon completion of this data pre-processing stage, the padded tokenized datasets are split into a training subset, a validation subset, and a testing subset.

Homomorphic encryption is then performed on each subset of the padded tokenized data, using the customer encryption scheme and the vendor encryption scheme as explained above. In a preferred approach, approximately 70% of the homomorphic encrypted datasets are used to train the model, and the remaining datasets are used in test and validation stages, as will be known to the skilled person. Of course, the subset sizes can be adjusted according to the total number of available datasets to further improve accuracy. It is only at this stage, i.e. after homomorphic encryption, that datasets may leave the facility. Since the data is no longer in a form that is human-readable, patient data security is ensured.

An embedding layer then maps the encrypted token representations into dense vector representations that preserve semantic relationships between the encrypted tokens. The resulting encrypted dense vectors are passed through an encrypted dense (fully-connected) layer with an activation function in the form of a rectified linear unit (ReLU). The ReLU introduces non-linearity into the model, allowing the model to capture complex relationships in the data. A softmax (normalized exponential function) activation is then applied to produce a probability distribution over multiple classes, i.e. predicting the likelihood of each word in the vocabulary being the next word in a sequence. The result is a probability distribution over the vocabulary.

The built model is then compiled with the homomorphic encrypted version of the categorical cross-entropy loss function and the homomorphic encrypted version of the Adam optimizer. Categorical cross-entropy is a known loss function for multiclass classification problems, which compares the predicted probability distribution with the actual distribution (typically a one-shot encoded vector), thereby allowing the model to determine its training progress. The Adam optimizer is a known optimization algorithm used to minimize the loss function during training.

The compiled homomorphic encrypted model is then trained with the homomorphic encrypted training data and homomorphic encrypted target labels to predict the next word given a sequence of words. During training, the input data comprises sequences of tokens (words or sub-words) from training data, and a target label is the token that immediately follows a sequence of the training text.

Training is carried out for a suitable number of epochs, and the result is a trained homomorphic encrypted model which, when fed with an appropriate text input, will synthesize a homomorphic encrypted medical imaging protocol. Training can be supervised, unsupervised, or self-supervised. A supervised training procedure preferably comprises a technique of backward-propagation to find the best set of parameters that will enable the HE-LLM to infer a ground truth from the input, in this case to infer the most suitable protocol entry to follow the preceding protocol entry. The training procedure is preferably configured to achieve a favourably low cost function. Of course, the HE-LLM can continue to learn by including any verified protocol into the training dataset.

The trained homomorphic encrypted model can then be deployed whenever a user requires a medical imaging protocol for an intended imaging task. To obtain an imaging protocol, the user prepares an initial input sequence or seed text. An input sequence can comprise multiple pairs of field descriptor and field value to characterize the patient (for example “PatientId: PD0001”; “Age: 45”; “Previous surgeries: None” etc.). Preferably, the protocol generation stage performs any necessary tokenization and padding on the input sequence, and performs homomorphic encryption on the tokenized and padded data. With this input, the trained homomorphic encrypted model (which may be running at an off-site location, on a cloud computing platform, etc.) creates a homomorphic encrypted text which is returned to the user at the facility. To convert the synthesized protocol into a useable—i.e. human-readable-protocol, a decryption stage then applies homomorphic decryption with the customer encryption scheme and the vendor encryption scheme to obtain a plain-text output protocol which is then presented to the user, for example on the monitor of a desktop computer. The fields of the synthesized protocol can be presented alongside the fields of a standard protocol, so that the user can readily identify any discrepancy between the values of these fields. For example, if parameter “T1-weighted TR” of the synthesized protocol suggests a value of 500 ms, and the corresponding field of the standard protocol gives a range of 400-800 ms, the user can be sure that the suggested value is acceptable.

### BRIEF DESCRIPTION OF THE DRAWINGS

Other objects and features of the present invention will become apparent from the following detailed descriptions considered in conjunction with the accompanying drawings. It is to be understood, however, that the drawings are designed solely for the purposes of illustration and not as a definition of the limits of the present invention.

FIG. 1 illustrates the principle of one or more embodiments of the present invention;

FIG. 2 is an exemplary flowchart of the inventive method;

FIG. 3 and FIG. 4 show a schematic representation of the inventive data processing arrangement;

FIG. 5 shows an exemplary imaging protocol synthesized using the inventive computer-implemented method.

In the diagrams, like numbers refer to like objects throughout. Objects in the diagrams are not necessarily drawn to scale.

### DETAILED DESCRIPTION

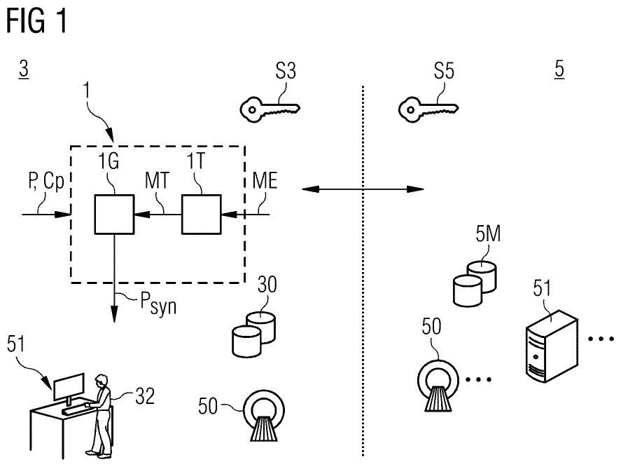

FIG. 1 illustrates the principle of one or more embodiments of the present invention. The diagram shows a facility 3, which can be a hospital, a clinic, a radiology practice, etc. The facility 3 can deploy any number of medical imaging devices 50 for imaging modalities such as CT, MRT, etc. Patient data is stored securely, for example in a PACS database 30. A vendor 5 supplies the facility 3 with software and hardware, for example with medical imaging devices 50 and software for operating the devices 50, a client-side server 51 for processing and analyzing medical images, and for managing the resulting data. The vendor 5 also provides software updates as necessary, and may also provide data processing services on a vendor-side server 51, a cloud computing service, etc.

Here, the vendor 5 also provides an AI model 5M. The homomorphic encrypted model ME is trained for the customer 3 using locally stored data P, CP, and the trained homomorphic encrypted model MT can then be used to generate medical imaging protocols Psyn for the customer as will be explained below.

Data security is ensured by deploying encryption at several stages during development and implementation of the computer-implemented method. Encryption is done using an encryption scheme S3 that is unique to the facility 3 and an encryption scheme S5 that is unique to the vendor 5.

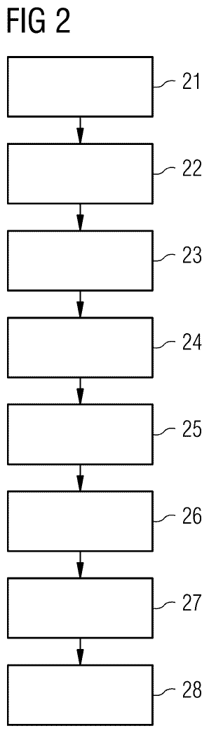

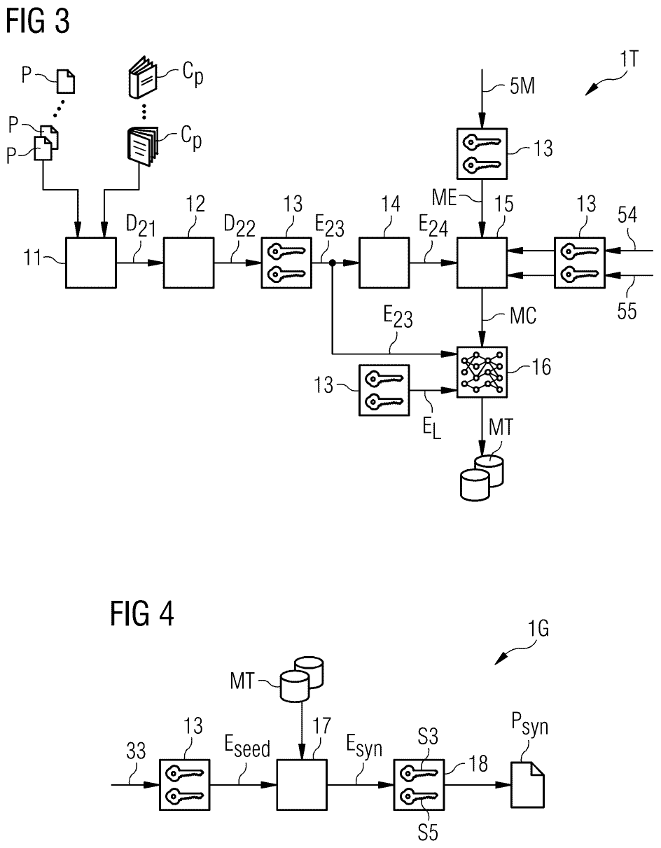

Units and modules of the data processing arrangement 1 can be distributed in any suitable configuration. In the configuration of FIG. 1, a model development stage 1T for training the model 5M and a protocol generation stage 1G for generating a protocol Esyn are realized at the customer site 3. Of course, the model development stage 1T can be realized at the vendor 5; equally, the protocol generation stage 1G can be realized at the vendor 5. FIGS. 2 and 3 show block diagrams to illustrate an exemplary embodiment of the inventive data processing apparatus 1.

An exemplary sequence of steps performed by the inventive method is illustrated in FIG. 2. Data collection is done in a first step 21. To this end, a collector stage 11 gathers a diverse and representative dataset of medical imaging protocols P for various medical conditions. These protocols P originate from the local facility 3. One or more local protocol cookbooks CP may also be collected. The collected data preferably includes a wide range of information such as patient demographics, clinical history, scan parameters, corresponding outcomes etc., and preferably covers a wide range of anatomical regions and pathologies.

A step of digitizing the information P, CP may also be performed at this stage if required. For example, a protocol cookbook in PDF format can be scanned and digitized to a suitable format such as XML markup language. The data D21 collected in this way comprises a collection of documents, i.e. a dataset of machine-readable protocols and, optionally, a dataset of machine-readable protocol cookbooks.

In a subsequent step 22, the collected datasets D21 are loaded into a pre-processing module 12, which performs tokenization and padding on the input data D21. During tokenization, the text of each document is converted into a sequence of tokens such as integers. To ensure that the tokenized sequences have the same length, padding is done as necessary. The data preprocessing step can also include a “cleaning” stage to remove duplicate entries or irrelevant entries. In this stage, a consistent formatting and structure can be applied to the data. Upon completion of the pre-processing step 22, the padded tokenized data D22 is split into a training subset, a validation subset, and a testing subset as explained above.

In a subsequent step 23, homomorphic encryption is applied by the encryption stage 13 to each subset of the padded tokenized data D22, using a homomorphic encryption scheme S3 unique to the facility 3 and a homomorphic encryption scheme S5 unique to the vendor 5 to obtain a homomorphic encrypted representation of the data E23.

In a subsequent step 24, an embedding layer 14 maps each homomorphic encrypted token from the homomorphic encrypted representation E23 to obtain a homomorphic encrypted dense vector representation E24.

In a subsequent step 25, homomorphic encryption is applied to each layer of the vendor-provided HE-LLM 5M. To build the HE-LLM model, the output of an encrypted layer is fed into an encrypted dense layer with rectified linear unit (ReLU) activation. A softmax (normalized exponential function) activation is then applied to predict the homomorphic encrypted probability distribution over the vocabulary for the next word. The model is then compiled with an encrypted version of the categorical cross-entropy loss function 54 and an encrypted version of the Adam optimizer 55.

In a subsequent step 26, a training stage 16 trains the compiled homomorphic encrypted model MC with homomorphic encrypted training data E23. Using plain text as an example, an input sequence might be “No evidence of cysts or tumors”. The corresponding tokens are [“No”, “evidence”, “of”, “cysts”, “or”, “tumors”], and possible training sequences can be [“No”, “evidence”, “of”]->Target: “cysts”; [“of”, “cysts”, “or”]->Target: “tumors”; etc. In the inventive approach, training the LLM is done exclusively on homomorphic encrypted data, and can therefore be done “off-site” without any risk of compromising patient data security. To this end, the homomorphic encrypted training data E23 and homomorphic encrypted target labels EL are passed to a fit function, for example a stochastic gradient descent function, a batch gradient descent function, a transfer learning function, etc. The model is trained for a suitable number of epochs. The output of this training stage 16 is a trained homomorphic encrypted model MT.

The generation and prediction of output text is addressed in a subsequent step 27, indicated by the predictor stage 17 in FIG. 4. Here, an input sequence 33 provided by the user undergoes (after being tokenized and padded as necessary) homomorphic encryption. The trained homomorphic encrypted model MT predicts the next word wpt based on the homomorphic encrypted input sequence Eseed. The seed text is updated by appending the predicted word. The process of generating predictions and updating the seed text is repeated until the desired number of words—i.e. the synthesized protocol Esyn—has been generated.

In a final step 28, the text Esyn obtained at the ultimate iteration is passed to a decryption stage 18, which applies homomorphic decryption (with site encryption scheme S3 and vendor encryption scheme S5) to obtain a plain-text synthesized output protocol Psyn which is then presented to the user, for example in tabular form.

All steps of building and training the model 5M can be done off-site, using homomorphic encrypted training datasets generated locally, i.e. at the hospital or clinic facility. Similarly, the trained model 5M can be tested and verified off-site using homomorphic encrypted test datasets and verification datasets. In this way, sensitive patient data remains local or on-site, while resource- and time-intensive model generation is outsourced, for example to a server farm (on-site or cloud-based) supplied by the vendor. The trained and verified model 5M—which can run at an offsite location such as on a cloud-computing server—can then be used at any time by a user of the facility, who need only provide basic relevant seed text that is homomorphic encrypted before leaving the facility. The model 5M returns a synthesized complete imaging protocol to the facility. The homomorphic encrypted protocol undergoes homomorphic decryption to make it human-readable.

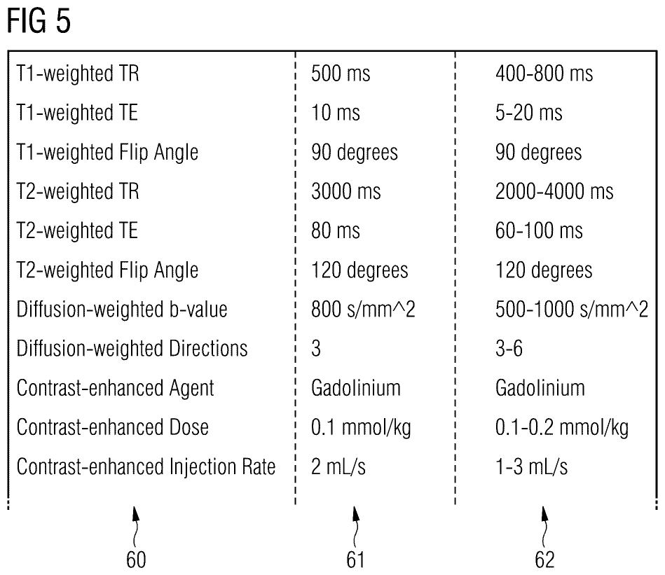

The protocol Psyn generated by the inventive data processing apparatus 1 can be presented alongside a standard protocol, so that the user can readily identify any potential discrepancy as illustrated in FIG. 5. The diagram shows a synthesized imaging protocol Psyn comprising a list of parameters 60 relevant to an imaging task, a list 61 of values as proposed in the synthesized imaging protocol Psyn, and a list 62 of corresponding allowable ranges as determined by the appropriate reference imaging protocol Pref (for example a cookbook protocol). The diagram shows that the values 61 in the synthesized imaging protocol Psyn are all within the allowed ranges 62 of the cookbook protocol Pref.

For the sake of clarity, it is to be understood that the use of “a” or “an” throughout this application does not exclude a plurality, and “comprising” does not exclude other steps or elements. The mention of a “unit” or a “module” does not preclude the use of more than one unit or module. Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been shown and described with respect to certain example embodiments, equivalents and modifications will occur to others skilled in the art upon the reading and understanding of the specification. The present invention includes all such equivalents and modifications and is limited only by the scope of the appended claims.

What is claimed is:

1. A computer-implemented method of generating a protocol for a medical imaging device installed at a specific site, the computer-implemented method comprising:
providing a homomorphic encrypted large-language model trained to generate a homomorphic encrypted medical imaging protocol;
obtaining patient-specific input data for an intended medical imaging task;
performing homomorphic encryption on the patient-specific input data;
applying the homomorphic encrypted patient-specific input data to the homomorphic encrypted large-language model to generate the homomorphic encrypted medical imaging protocol; and
performing homomorphic decryption on the generated homomorphic encrypted medical imaging protocol to obtain a plain-text medical imaging protocol; wherein
the homomorphic encryption and the homomorphic decryption utilize a first encryption scheme specific to the specific site and a second encryption scheme specific to a vendor.

2. The computer-implemented method according to claim 1, further comprising:
presenting the plain-text medical imaging protocol to a user.

3. The computer-implemented method according to claim 1, wherein the homomorphic encrypted large-language model is provided by the vendor.

4. A computer-implemented method of training a large-language model for use in the computer-implemented method according to claim 1, wherein the computer-implemented method of training comprises:
modifying a large-language model to include a homomorphic encrypted layer architecture;
obtaining homomorphic encrypted training data based on imaging protocols for the medical imaging device installed at the specific site; and
training the homomorphic encrypted large-language model on the homomorphic encrypted training data to generate a homomorphic encrypted medical imaging protocol.

5. The computer-implemented method according to claim 4, wherein the homomorphic encrypted training data is generated by obtaining protocol data for the medical imaging device installed at the specific site;
generating training data from the protocol data; and
performing homomorphic encryption on the training data using the first encryption scheme and the second encryption scheme.

6. The computer-implemented method according to claim 4, wherein the imaging protocols include at least several hundred imaging protocols.

7. The computer-implemented method according to claim 4, wherein protocol data for the imaging protocols includes a number of imaging protocol cookbooks for the medical imaging device.

8. The computer-implemented method according to claim 4, wherein the computer-implemented method of training is performed by the vendor.

9. The computer-implemented method according to claim 1, wherein the homomorphic encrypted large-language model is trained by a computer-implemented method comprising:
modifying a large-language model to include a homomorphic encrypted layer architecture;
obtaining homomorphic encrypted training data based on imaging protocols for the medical imaging device installed at the specific site; and
training the homomorphic encrypted large-language model on the homomorphic encrypted training data to generate a homomorphic encrypted medical imaging protocol.

10. A data processing system to perform the computer-implemented method according to claim 1, the data processing system comprising:
an encryption module configured to perform the homomorphic encryption on the patient-specific input data for the intended medical imaging task;
a protocol generation stage configured to apply the homomorphic encrypted patient-specific input data to the homomorphic encrypted large-language model to obtain the homomorphic encrypted medical imaging protocol; and
a decryption module configured to perform the homomorphic decryption on the homomorphic encrypted medical imaging protocol to obtain the plain-text medical imaging protocol.

11. The data processing system according to claim 10, wherein the encryption module and the decryption module are located at the specific site.

12. A data processing system according to claim 10, wherein the protocol generation stage is provided by the vendor.

13. A data processing system according to claim 10, further comprising:
a model development stage provided by the vendor, the model development stage including preparatory stages configured to prepare homomorphic encrypted training data from collected imaging protocol data.

14. A data processing system according to claim 13, wherein the model development stage includes further stages configured to train a homomorphic encrypted version of the large-language model with the homomorphic encrypted training data.

15. A non-transitory computer-readable storage medium storing computer-executable instructions that, when executed by a computer, cause the computer to perform the computer-implemented method of claim 1.

16. A computer-implemented method of training a large-language model for use in the computer-implemented method according to claim 2, wherein the computer-implemented method of training comprises:
modifying a large-language model to include a homomorphic encrypted layer architecture;
obtaining homomorphic encrypted training data based on imaging protocols for the medical imaging device installed at the specific site; and
training the homomorphic encrypted large-language model on the homomorphic encrypted training data to generate a homomorphic encrypted medical imaging protocol.

17. The computer-implemented method according to claim 5, wherein the protocol data includes a number of imaging protocol cookbooks for the medical imaging device.

18. The computer-implemented method according to claim 2, wherein the homomorphic encrypted large-language model is trained by a computer-implemented method comprising:
modifying a large-language model to include a homomorphic encrypted layer architecture;
obtaining homomorphic encrypted training data based on imaging protocols for the medical imaging device installed at the specific site; and
training the homomorphic encrypted large-language model on the homomorphic encrypted training data to generate a homomorphic encrypted medical imaging protocol.

19. A data processing system to perform the computer-implemented method according to claim 2, the data processing system comprising:
an encryption module configured to perform the homomorphic encryption on the patient-specific input data for the intended medical imaging task;
a protocol generation stage configured to apply the homomorphic encrypted patient-specific input data to the homomorphic encrypted large-language model to obtain the homomorphic encrypted medical imaging protocol; and
a decryption module configured to perform the homomorphic decryption on the homomorphic encrypted medical imaging protocol to obtain the plain-text medical imaging protocol.

20. A data processing system comprising:
a memory storing computer-executable instructions; and
at least one processor configured to execute the computer-executable instructions to cause the data processing system to
provide a homomorphic encrypted large-language model trained to generate a homomorphic encrypted medical imaging protocol,
obtain patient-specific input data for an intended medical imaging task,
perform homomorphic encryption on the patient-specific input data,
applying the homomorphic encrypted patient-specific input data to the homomorphic encrypted large-language model to generate the homomorphic encrypted medical imaging protocol, and
perform homomorphic decryption on the homomorphic encrypted medical imaging protocol to obtain a plain-text medical imaging protocol, wherein
the homomorphic encryption and the homomorphic decryption utilize a first encryption scheme specific to a site and a second encryption scheme specific to a vendor.
